(19) United States
(12) Patent Application Publication
(10) Pub. No.: US 2002/0002377 A1
(43) Pub. Date: Jan. 3, 2002

(54) Aluminum ultrasonic surgical applicator and method of making such an applicator

(76) Inventor: William W. Cimino, Louisville, CO (US)

Correspondence Address: MERCHANT & GOULD PC, P.O. BOX 2903, MINNEAPOLIS, MN, 55402-0903, US

(21) Appl. No.: 09/773,454

(22) Filed: Jan. 31, 2001

Related U.S. Application Data
(60) Provisional application No. 60/179,494, filed on Feb. 1, 2000.

(51) Int. Cl. A61B 17/32

(52) U.S. Cl. 606/169

(57) ABSTRACT

An applicator, such as a probe, tip or blade, for an ultrasonic surgical device, the applicator being shaped and sized for surgical application, and comprising: (a) a base material forming the applicator, the base material being a high-strength aluminum alloy; and (b) a surface coating on the applicator, the surface coating being aluminum oxide, and said surface coating having a thickness between 0.0001 and 0.0003 inch.

This application claims the benefit of the filing date of U.S. provisional patent application Serial No. 60/179,494, filed Feb. 1, 2000.

### FIELD OF THE INVENTION

This invention relates to the field of ultrasonic surgical instruments and, more specifically, to the materials from which the ultrasonic applicators of such instruments are made.

### BACKGROUND

Ultrasonic surgical instruments that utilize ultrasonic frequency vibrations to achieve a surgical effect have been in existence for more than 40 years. For example, U.S. Pat. No. 3,086,288 describes an ultrasonically vibrated cutting knife, U.S. Pat. No. 3,794,040 describes a method and apparatus that uses ultrasonic frequency vibrations to close off blood vessels, and U.S. Pat. No. 4,886,060 describes an apparatus that combines suction and irrigation with an ultrasonically vibrated knife. Numerous other U.S. patents describe technology for ultrasonic surgical devices and methods, including those used in ultrasonic assisted lipoplasty.

Ultrasonic surgical instruments typically vibrate at frequencies between 20 kHz and 60 kHz. Each ultrasonic surgical instrument has an ultrasonic applicator that is placed into contact with the tissues of a patient to cause a surgical effect. For example, the “applicator” may include the portion of the ultrasonic surgical instrument known as the “probe,” “tip” or “blade.”

Patents have disclosed the use of various materials for forming ultrasonic applicators, including titanium and titanium alloys, stainless steel, and aluminum. For example, U.S. Pat. No. 5,419,761 discloses ultrasonic applicators fabricated from titanium and aluminum. U.S. Pat. No. 3,990,452 discloses ultrasonic applicators fabricated from stainless steel or titanium.

In practice, however, the applicators of ultrasonic surgical devices are almost universally fabricated from titanium or titanium alloys, most often Ti6Al4V. This titanium alloy is used because of its excellent fatigue properties, good ultimate strength (typically about 130 ksi as reported in Titanium, Appendix 1, by Titanium Industries, 110 Lehigh Drive, Fairfield, N.J. 07004), good surface hardness (typically about Rc36 as reported in the same source), low internal losses, and inherent biocompatibility. Titanium and various titanium alloys are implant grade metals and are considered to be among the most biocompatible metals available today.

In contrast, stainless steel and aluminum are not used as applicators of ultrasonic surgical devices. Although stainless steel has been used in the past, it has significant drawbacks. Among other things, stainless steel “self-heats” (meaning that stresses that occur due to vibratory expansion and contraction of the material cause the metal to heat up) more rapidly than titanium or titanium alloys. Stainless steel also tends to have a shorter lifetime before failure. Aluminum and aluminum alloys have not been used in commercially available applicators because of their lower fatigue strengths, lower surface hardness, and general lack of biocompatibility. It would be highly desirable to use aluminum or aluminum allows in such surgical instruments because these materials are much less expensive than titanium and its alloys and are much cheaper to machine into finished instruments.

The present invention provides an ultrasonic applicator fabricated essentially of aluminum alloy that has sufficient fatigue strength, a sufficiently hard surface to withstand typical surgical applications, and biocompatibility characteristics that allow it to be used in a surgical environment.

### SUMMARY OF THE INVENTION

As described more fully herein, the present invention comprises an applicator for an ultrasonic surgical device, the applicator being shaped and sized for surgical application, and comprising: (a) a base material forming the applicator, the base material being a high-strength aluminum alloy; and (b) a surface coating on the applicator, the surface coating being aluminum oxide, and the surface coating having a thickness between about 0.0001 and 0.0003 inch.

The invention also includes a method of making an applicator for an ultrasonic surgical device, the method comprising: (a) fabricating an applicator from a high-strength aluminum alloy and (b) coating the surface of the applicator with aluminum oxide, the thickness of the coating being between about 0.0001 and 0.0003 inch.

### DETAILED DESCRIPTION OF THE INVENTION AND THE PREFERRED EMBODIMENT

The current invention enables the use of an aluminum alloy applicator for ultrasonic surgical applications by providing an outer surface coating that has improved hardness and meets the requisite biocompatibility requirements. Such a surface can be provided using aluminum oxide (Al₂O₃) in specific thicknesses.

Aluminum oxide is a brittle ceramic. Normally if aluminum oxide were used in or on an ultrasonic applicator, it would easily crack as the applicator extends and contracts during vibration, resulting in a decrease in fatigue strength and increasing the potential for fracture of the applicator. Thus, simply coating an ultrasonic applicator with aluminum oxide does not meet the requirements for such a device. Further complicating matters, aluminum oxide coatings will tend to “craze,” i.e., form small cracks, when subjected to autoclave temperatures in a steam environment—conditions normally employed in the use or reuse of an applicator for an ultrasonic surgical device. Thus, it would not be expected that aluminum oxide could be employed successfully as a coating for an applicator of an ultrasonic surgical device. One would not expect that the combination of an aluminum alloy and an aluminum oxide coating could provide an applicator for an ultrasonic surgical device with the requisite fatigue strength, surface hardness, and biocompatibility.

It has now been found that an applicator for an ultrasonic surgical device can be fabricated with a core of aluminum alloy and a thin coating of aluminum oxide. The requisites for an acceptable applicator can be achieved, if the thickness of the aluminum oxide coating is properly controlled. Specifically, if the coating is “clear,” i.e., having no dye or color additives, the thickness of the aluminum oxide coating should be controlled to between about 0.0001 and 0.0003 inch, preferably between 0.0001 and 0.0002 inch. If the coating is less than about 0.0001 inch, it will not provide sufficient biocompatibility. If the coating is thicker than about 0.0005 inch, the coating will have an increased tendency to crack and thereby decrease fatigue strength and increase the potential for fracture. If a dye or colorant is included in the coating, its thickness should be between about 0.0003 and 0.0005 inch, preferably between 0.0003 and 0.0004 inch. The coating should be thicker when it contains a dye or colorant because the color will not be visible if the coating is too thin. The thinner clear coating is preferred over the thicker colored coating for this reason.

The aluminum oxide coating is applied over a base of aluminum alloy. Pure aluminum does not have the requisite strength properties for use as in an applicator of an ultrasonic surgical device. However, high strength aluminum alloys, such as A16061 and A17075, provide an acceptable base. The A17075 alloy is the preferred alloy, having the highest strength characteristics of the aluminum alloy family.

An aluminum alloy ultrasonic applicator with an aluminum oxide coating can be manufactured in two basic steps. First, the aluminum alloy ultrasonic applicator is prepared from the appropriate stock, e.g., a tube of the requisite dimensions, and machined to the desired shape or profile, typically using a turning lathe. Mill work may also be required to form flats, cut-outs, beveled edges, or other required shapes. Second, the machined aluminum alloy ultrasonic applicator is cleaned and then coated with aluminum oxide, typically in a controlled anodizing process in which the parameters that control the coating thickness (e.g., time of exposure, voltage, current, and/or concentration) are regulated to supply an aluminum oxide coating of the appropriate thickness. The precise values for the control parameters may vary with the size and shape of the applicator, but are generally known to or can easily be determined by one skilled in the art. Preferably anodizing is conducted is accordance with Mil. Std. A2685. More preferably the anodizing is Type II, Class 1.

### EXAMPLE

An ultrasonic fragmentation probe was prepared in the configuration shown in FIG. 1 using a base material aluminum alloy of A17075. The probe was anodized by a commercial anodizing supplier using conditions generally employed in providing a very thin aluminum oxide coating of approximately 0.00015 inch. The resulting probe was vibrated ultrasonically at 36 kHz in water and showed fragmentation capabilities on biological materials such as oranges and grapefruit. An examination of the probe showed no cracking, breaking, crazing or chipping of the aluminum oxide surface after being operated under these conditions. The probe was autoclaved. There were no visible changes, i.e., crazing, to the surface of the probe.

1. An ultrasonic applicator for an ultrasonic surgical device, the applicator being shaped and sized for surgical application, the applicator comprising:
a base portion comprising a high-strength aluminum alloy material; and
a surface coating of aluminum oxide having a thickness between about 0.0001 and 0.0003 inch.

2. The ultrasonic applicator of claim 1 wherein the applicator is a probe, tip or blade of an ultrasonic surgical device.

3. The ultrasonic applicator of claim 1 wherein the ultrasonic surgical device is a lipoplasty device.

4. The ultrasonic applicator of claim 1 wherein the aluminum alloy is a member of the group consisting of A16061 and A17075.

5. The ultrasonic applicator of claim 1 wherein the surface coating of aluminum oxide has a thickness of between about 0.0001 and 0.0002 inch.

6. The ultrasonic applicator of claim 1 wherein the surface coating of aluminum oxide includes a dye or colorant and the thickness of the surface coating is between 0.0003 and 0.0005 inch.

7. The ultrasonic applicator of claim 6 wherein the surface coating of aluminum oxide has a thickness between about 0.0003 and 0.0004 inch.

8. An ultrasonic applicator for an ultrasonic lipoplasty surgical device, the applicator being shaped and sized for surgical application, the applicator comprising:
a base portion comprising a high-strength aluminum alloy material selected from the group consisting of A16061 and A17075; and
a surface coating of aluminum oxide having a thickness between about 0.0001 and 0.0003 inch.

9. The ultrasonic applicator of claim 8 wherein the surface coating of aluminum oxide has a thickness of between about 0.0001 and 0.0002 inch.

10. The ultrasonic applicator of claim 8 wherein the surface coating of aluminum oxide includes a dye or colorant and the thickness of the surface coating is between 0.0003 and 0.0005 inch.

11. The ultrasonic applicator of claim 10 wherein the surface coating of aluminum oxide has a thickness between about 0.0003 and 0.0004 inch.

12. An ultrasonic blade for an ultrasonic surgical cutting device, the applicator being shaped and sized for surgical application, the blade comprising:
a base portion comprising a high-strength aluminum alloy material selected from the group consisting of A16061 and A17075; and
a surface coating of aluminum oxide having a thickness between about 0.0001 and 0.0003 inch.

13. The ultrasonic blade of claim 12 wherein the surface coating of aluminum oxide has a thickness of between about 0.0001 and 0.0002 inch.

14. The ultrasonic blade of claim 12 wherein the surface coating of aluminum oxide includes a dye or colorant and the thickness of the surface coating is between 0.0003 and 0.0005 inch.

15. The ultrasonic blade of claim 14 wherein the surface coating of aluminum oxide has a thickness between about 0.0003 and 0.0004 inch.

16. A method of making an ultrasonic applicator for an ultrasonic surgical device comprising:
fabricating an ultrasonic applicator from a high-strength aluminum alloy; and
coating the surface of the ultrasonic applicator with aluminum oxide, the thickness of the coating between about 0.0001 and 0.0003 inch.

17. The method of claim 16 wherein the applicator is a probe, tip or blade of an ultrasonic surgical device.

18. The method of claim 16 wherein the ultrasonic surgical device is a lipoplasty device.

19. The method of claim 16 wherein the aluminum alloy is a member of the group consisting of A16061 and A17075.

20. The method of claim 16 wherein the surface coating of aluminum oxide has a thickness of between about 0.0001 and 0.0002 inch.

21. The method of claim 16 wherein the surface coating of aluminum oxide includes a dye or colorant and the thickness of the surface coating is between 0.0003 and 0.0005 inch.

22. The method of claim 21 wherein the surface coating of aluminum oxide has a thickness between about 0.0003 and 0.0004 inch.

23. The method of claim 16 wherein the coating is performed by anodizing.

24. A method of using an ultrasonic applicator for an ultrasonic surgical devise comprising:
applying an ultrasonic applicator of an ultrasonic surgical device to the tissues of a patient, the ultrasonic applicator being fabricated from a high-strength aluminum alloy and having a coating on its surface of aluminum oxide, the coating having a thickness between about 0.0001 and 0.0003 inch; and
vibrating the ultrasonic applicator at an operating resonant frequency to achieve a surgical effect.

25. The method of claim 24 wherein the applicator is a probe, tip or blade of an ultrasonic surgical device.

26. The method of claim 24 wherein the ultrasonic surgical device is a lipoplasty device.

27. The method of claim 24 wherein the aluminum alloy is a member of the group consisting of A16061 and A17075.

28. The method of claim 24 wherein the surface coating of aluminum oxide has a thickness of between about 0.0001 and 0.0002 inch.

29. The method of claim 24 wherein the surface coating of aluminum oxide includes a dye or colorant and the thickness of the surface coating is between 0.0003 and 0.0005 inch.

30. The method of claim 29 wherein the surface coating of aluminum oxide has a thickness between about 0.0003 and 0.0004 inch.

31. The method of claim 24 wherein the coating is performed by anodizing.
